(19) United States
(12) Patent Application Publication
(10) Pub. No.: US 2022/0347345 A1
(43) Pub. Date: Nov. 3, 2022

(54) BIOADHESIVE SHEET-SHAPED MATERIAL FOR ATTACHING ONTO SURFACE OF ORGAN

(71) Applicants: NAGASAKI UNIVERSITY, Nagasaki-shi (JP); TERUMO KABUSHIKI KAISHA, Tokyo (JP)

(72) Inventors: Yasuhiro MARUYA, Nagasaki-shi (JP); Shun YAMAGUCHI, Nagasaki-shi (JP); Kengo KANETAKA, Nagasaki-shi (JP); Miki HIGASHI, Nagasaki-shi (JP); Susumu EGUCHI, Nagasaki-shi (JP); Fumiya OHASHI, Ashigarakami-gun (JP); Masaki MATSUMURA, Ashigarakami-gun (JP)

(73) Assignees: NAGASAKI UNIVERSITY, Nagasaki-shi (JP); TERUMO KABUSHIKI KAISHA, Tokyo (JP)

(21) Appl. No.: 17/869,392

(22) Filed: Jul. 20, 2022

Related U.S. Application Data
(63) Continuation of application No. PCT/JP2021/002375, filed on Jan. 25, 2021.

(30) Foreign Application Priority Data
Jan. 24, 2020 (JP) 2020-010071

(51) Int. Cl. A61L 27/36; C12N 5/077; A61L 27/58; A61L 27/38; A61L 27/54

(52) U.S. Cl. CPC A61L 27/3633; C12N 5/0658; A61L 27/58; A61L 27/3826; A61L 27/3834; A61L 27/54; A61L 27/3687; C12N 2533/56

(57) ABSTRACT

A bioadhesive sheet-shaped material configured to be attached onto a surface of an organ, a method for producing the bioadhesive sheet-shaped material, and a method for treating a disease by using the bioadhesive sheet-shaped material. The bioadhesive sheet-shaped material includes an extracellular matrix layer, a sheet-shaped cell culture, and a biodegradable gel layer, where the sheet-shaped cell culture is interposed between the extracellular matrix layer and the biodegradable gel layer, and the bioadhesive sheet-shaped material is by attaching the extracellular matrix layer onto a surface of an organ.

### CROSS-REFERENCES TO RELATED APPLICATIONS

This application is a continuation of International Application No. PCT/JP2021/002375 filed on Jan. 25, 2021, which claims priority based on Japanese Patent Application No. 2020-010071 filed on Jan. 24, 2020, the entire content of both of which is incorporated herein by reference.

### TECHNOLOGICAL FIELD

The present disclosure relates to a bioadhesive sheet-shaped material including an extracellular matrix layer, a sheet-shaped cell culture, and a biodegradable gel layer, in which the bioadhesive sheet-shaped material has the extracellular matrix layer on one surface and the biodegradable gel layer on the other surface with the sheet-shaped cell culture interposed therebetween. The bioadhesive sheet-shaped material is used by attaching the extracellular matrix layer onto a surface of an organ. The disclosure also relates to a method for producing the bioadhesive sheet-shaped material, and a method for treating a disease by using the bioadhesive sheet-shaped material.

### BACKGROUND DISCUSSION

In recent years, in order to repair damaged tissues or the like, attempts to transplant various cells have been made. For example, for repairing myocardial tissues damaged due to an ischemic heart disease such as angina pectoris or myocardial infarction, attempts to utilize fetal cardiomyocytes, skeletal myoblast cells, mesenchymal stem cells, cardiac stem cells, embryonic stem (ES) cells, induced pluripotent stem (iPS) cells, and the like have been made (Haraguchi et al., Stem Cells Trans1 Med. 2012 February; 1(2): 136-41).

As an example of such attempts, sheet-shaped cell cultures, which are obtained by forming cells into a sheet shape, have been developed, and some of the sheet-shaped cell cultures are in the stage of clinical application. Examples of such clinical application include repairing impaired myocardium by implanting engineered autologous myoblast sheets (Imran A. Memon et al., The Journal of Thoracic and Cardiovascular Surgery, Volume 130, Number 5, 1333-1341), attaching a cell sheet containing liver cells derived from mesenchymal stem cells onto a liver surface for the purpose of suppressing liver damage (Japanese Patent Publication No. 60082972 (JP60082972)), reducing liver fibrosis by transplanting IC-2-engineered mesenchymal stem cell sheet (Noriko Itaba et al., SCIENTIFIC REPORT (2019) 9:6841), and a cell sheet composition for curing or preventing the leakage from a wound site of a hollow organ by attaching the cell sheet composition at the wound site of the hollow organ (International Patent Application Publication No. 2017/130802 (WO2017/130802)).

Further, a sheet-shaped cell culture is fragile, and thus, it is difficult to use the sheet-shaped cell culture in operation in clinical application. For this reason, for example, a sheet-shaped cell culture having favorably improved adhesiveness to tissue (International Patent Application Publication No. 2006/093153 (WO2006/093153)), a laminate of a fibrin gel and a sheet-shaped cell culture (Japanese Patent Publication No. 6495603 (JP 6495603 B2)), and the like have been proposed.

### SUMMARY

Recognizing that development of a new sheet-shaped cell culture with higher operability is required for the further spread of regenerative medicine, the present disclosure provides a bioadhesive sheet-shaped material having favorable bioadhesiveness onto a surface of an organ which is easy to operate in clinical application, a method for producing the bioadhesive sheet-shaped material, and a method for treating a disease by using the bioadhesive sheet-shaped material.

In conducting intensive studies on the sheet-shaped cell culture that is engrafted well into various tissues, the present inventors have found for the first time that a myoblast sheet having an extracellular matrix layer on one surface and a biodegradable gel layer on the other surface can be engrafted well onto a surface of liver or large intestine by attaching a surface of the extracellular matrix layer to an organ, and as a result of further studies based on such a finding, the present inventors have completed the present disclosure.

The present disclosure relates to the following:

[1] A bioadhesive sheet-shaped material including: an extracellular matrix layer; a sheet-shaped cell culture; and a biodegradable gel layer, wherein the bioadhesive sheet-shaped material has the extracellular matrix layer on one surface and the biodegradable gel layer on the other surface with the sheet-shaped cell culture interposed therebetween, which can be used by attaching the extracellular matrix layer onto a surface of an organ.

[2] The bioadhesive sheet-shaped material described in [1], in which the organ is a human organ.

[3] The bioadhesive sheet-shaped material described in [1] or [2], in which the attachment onto a surface of an organ is performed under a laparoscope or a thoracoscope.

[4] The bioadhesive sheet-shaped material described in any one of [1] to [3], in which the sheet-shaped cell culture contains somatic cells.

[5] The bioadhesive sheet-shaped material described in [4], in which the somatic cells are myoblast cells or mesenchymal stem cells.

[6] The bioadhesive sheet-shaped material described in any one of [1] to [5], in which the organ is an organ in the body, capable of being approached from outside the body by a laparoscope or a thoracoscope.

[7] The bioadhesive sheet-shaped material described in any one of [1] to [6], further including a medical component.

[8] A method for producing the bioadhesive sheet-shaped material includes: seeding cells on a substrate; forming the seeded cells into a sheet shape; and detaching the formed sheet-shaped cell culture from the substrate.

[9] A method for treating a disease of a subject that is ameliorated by application of a bioadhesive sheet-shaped material includes attaching the bioadhesive sheet-shaped material to an organ of the subject, the bioadhesive sheet-shaped material comprising an extracellular matrix layer, a sheet-shaped cell culture and a biodegradable gel layer, the extracellular matrix layer being one surface of the bioadhesive sheet-shaped material and the biodegradable gel layer being an opposite surface of the bioadhesive sheet-shaped material, with the sheet-shaped cell culture being interposed between the extracellular matrix layer and the biodegradable gel layer. The attaching of the bioadhesive sheet-shaped material to the organ of the subject includes attaching the extracellular matrix layer of the bioadhesive sheet-shaped material in an effective amount to the organ of the subject.

[10] The method described in [9], in which the attachment to the organ of a subject is performed under a laparoscope or under a thoracoscope.

[11] The method described in [9] or [10], in which the attachment to an organ of a subject is performed by using a device including: a support member for supporting a bioadhesive sheet-shaped material; and a protective member with a low coefficient of friction for protecting one surface of the support, in which the support supporting the bioadhesive sheet-shaped material is protected with the protective member, the protected support is rolled up, and the rolled-up support is inserted into a cylindrical body.

By using the bioadhesive sheet-shaped material of the present disclosure, favorable bioadhesiveness can be obtained when the bioadhesive sheet-shaped material containing a sheet-shaped cell culture is attached to an organ of a subject. Further, the bioadhesive sheet-shaped material of the present disclosure can be more easily attached to an organ of a subject. In addition, in the attachment of the bioadhesive sheet-shaped material of the present disclosure to an organ of a subject, a device for delivery can be used. Additionally, by a less invasive method, for example, a method of using a laparoscope, the bioadhesive sheet-shaped material can be attached to an organ of a subject. Moreover, the organ after the attachment of the bioadhesive sheet-shaped material can retain a favorable condition.

### BRIEF DESCRIPTION OF THE DRAWINGS

FIGS. 1A and 1B each show a stained tissue image obtained by staining a tissue, to which a bioadhesive sheet-shaped material has been attached, from the stomach with hematoxylin and eosin (HE). The part surrounded by a circle with a dashed line shows the attached bioadhesive sheet-shaped material. The part surrounded by a circle with a straight line shows a part indicating histological continuity. FIG. 1B shows a further enlarged part of FIG. 1A.

FIGS. 2A and 2B each show a stained tissue image obtained by staining a tissue, to which a bioadhesive sheet-shaped material has been attached, from the large intestine with hematoxylin and eosin. FIG. 2B shows a further enlarged part of FIG. 2A.

FIGS. 2C and 2D each show a stained tissue image obtained by immunostaining a tissue, to which a bioadhesive sheet-shaped material has been attached, from the large intestine with the use of an anti-desmin antibody. FIG. 2D shows a further enlarged part of FIG. 2C.

FIGS. 3A and 3B each show a stained tissue image obtained by staining a tissue, to which a bioadhesive sheet-shaped material has been attached, from the liver with Azan. FIG. 3B shows a further enlarged part of FIG. 3A. The arrows in the diagram show parts where histological continuity is remarkably observed.

FIGS. 3C and 3D each show a stained tissue image obtained by immunostaining a tissue, to which a bioadhesive sheet-shaped material has been attached, from the liver with the use of an anti-desmin antibody. FIGS. 3C and 3D show images in different fields of view within the same slide. The arrows in the diagram show parts where histological continuity is remarkably observed. Further, a bioadhesive sheet-shaped material was attached to the side expressed by “Sheet” in the diagram.

### DETAILED DESCRIPTION

The present disclosure relates to a bioadhesive sheet-shaped material including an extracellular matrix layer, a sheet-shaped cell culture, and a biodegradable gel layer, in which the bioadhesive sheet-shaped material has the extracellular matrix layer on one surface and the biodegradable gel layer on the other surface with the sheet-shaped cell culture interposed therebetween. The bioadhesive sheet-shaped material can be used by attaching the extracellular matrix layer onto a surface of an organ.

The expression “bioadhesive sheet-shaped material” refers to a sheet-shaped material that can be attached to a subject with favorable bioadhesiveness. The bioadhesive sheet-shaped material of the present disclosure includes an extracellular matrix layer, a sheet-shaped cell culture, and a biodegradable gel layer, and has the extracellular matrix layer on one surface of the sheet-shaped cell culture contained in the bioadhesive sheet-shaped material and the biodegradable gel layer on the other surface with the sheet-shaped cell culture interposed therebetween.

The expression “favorable bioadhesiveness” means, for example, that a bioadhesive sheet-shaped material and an organ to which the bioadhesive sheet-shaped material has been attached are histologically continuous with each other. The expression “a bioadhesive sheet-shaped material and an organ are histologically continuous with each other” means that a bioadhesive sheet-shaped material and an organ are continuously in contact with each other without leaving any space between them. In this regard, the expression “without leaving any space between them” means that a bioadhesive sheet-shaped material and an organ are at least partially in contact with each other without forming any space between them. For example, in order to eliminate a space between a bioadhesive sheet-shaped material and an organ, the bioadhesive sheet-shaped material and the organ may be sutured. In a case where there is a space between a bioadhesive sheet-shaped material and an organ, other cells can infiltrate the space. The sheet-shaped cell culture in the bioadhesive sheet-shaped material can provide a perforation prevention effect, a liquid leakage prevention effect from the organ, a therapeutic effect via reducing fibrosis, and the like. As to the bioadhesive sheet-shaped material of the present disclosure, since an extracellular matrix layer (also referred to as ECM) is attached onto a surface of an organ, there is the extracellular matrix layer between the sheet-shaped cell culture in the bioadhesive sheet-shaped material and the organ. In a part where the bioadhesive sheet-shaped material is in contact with an organ, it can be considered that cells composing the bioadhesive sheet-shaped material migrate into the organ, or on the contrary, cells composing the organ enter the bioadhesive sheet-shaped material. At this time, it can be considered that cells enter and migrate through the extracellular matrix.

The expression “extracellular matrix layer” refers to a layer obtained by forming an extracellular matrix in a layer form. The extracellular matrix layer contributes to the adhesion of a bioadhesive sheet-shaped material to an organ of a subject and improves the survival rate of the bioadhesive sheet-shaped material at an attachment site when the bioadhesive sheet-shaped material is attached. Further, the extracellular matrix is a cell-derived component, and thus there is no problem in using the bioadhesive sheet-shaped material of the present disclosure for attachment to an organ of a subject together with the contained extracellular matrix layer. The thickness of the extracellular matrix layer can be from 1 nm to 100 nm, and preferably 1 nm to 50 nm.

The expression “sheet-shaped cell culture” means a cell culture in a sheet shape formed by connecting cells to each other. The cells may be connected to each other directly (including connection through a cellular element such as an adhesion molecule) and/or through a mediator. The mediator is not particularly limited as long as it is a substance that can at least physically (mechanically) connect cells to each other, and as the mediator, for example, an extracellular matrix or the like can be mentioned. The mediator is preferably derived from a cell, and particularly derived from a cell that forms a cell culture. The cells are at least physically (mechanically) connected to each other, but may be more functionally, for example, chemically electrically connected to each other. The sheet-shaped cell culture may be formed of one cell layer (single layer), or may also be formed of two or more cell layers (such as a laminated (multilayer) body, for example, two-layer, three-layer, four-layer, five-layer, or six-layer). Further, the sheet-shaped cell culture may have a three-dimensional structure having a thickness exceeding the thickness of one cell without showing any clear layered structure of the cells. For example, in the vertical section of the sheet-shaped cell culture, the cells may be present in a state of being non-uniformly (for example, mosaic-like) arranged without being uniformly aligned in the horizontal direction.

The sheet-shaped cell culture may not contain a scaffold (support). A scaffold may be used in some cases, in the technical field to which the present disclosure belongs, in order to attach cells onto the surface of and/or to the inside of the scaffold and to maintain the physical integrity of the sheet-shaped cell culture. The scaffold, for example, may be a membrane made of polyvinylidene difluoride (PVDF) or a known similar material. The sheet-shaped cell culture contained in the bioadhesive sheet-shaped material of the present disclosure can maintain the physical integrity even without such a scaffold. Further, the sheet-shaped cell culture contained in the bioadhesive sheet-shaped material of the present disclosure may consist only of cell-derived substances making up the sheet-shaped cell culture, and does not contain any other substances.

The thickness of the sheet-shaped cell culture is not particularly limited. In a case where a single-layer sheet is used as the sheet-shaped cell culture, the thickness is usually a thickness of one or more cells, and varies depending on the kind of cells forming the sheet-shaped cell culture. In one embodiment, the sheet-shaped cell culture has a thickness of 30 μm or more, and in another embodiment, the sheet-shaped cell culture has a thickness of 50 μm or more. Examples of the range of thickness values the sheet-shaped cell culture may possess include 30 μm to 200 μm, preferably 50 μm to 150 μm, and more preferably 60 μm to 100 μm. In a case where a laminated sheet is used as the sheet-shaped cell culture, the thickness does not exceed the value obtained by a thickness of the single-layer sheet×the number of laminated sheets. Accordingly, as one embodiment, in a case where a sheet obtained by stacking five single-layer sheets in layers is used, the sheet has a thickness of 150 μm or more, and in one preferred embodiment, the sheet has a thickness of 250 μm or more. In these cases, examples of the range of the thickness value of the sheet-shaped cell culture include 150 μm to 1000 μm, preferably 250 μm to 750 μm, and more preferably 300 μm to 500 μm.

The cells contained in the bioadhesive sheet-shaped material can be derived from any organism used for attachment of the bioadhesive sheet-shaped material, and examples of the organism include, but are not limited to, a human, primates, a dog, a cat, a pig, a horse, a goat, a sheep, a rodent animal (for example, a mouse, a rat, a hamster, or a guinea pig), and a rabbit. Further, as the cells contained in the bioadhesive sheet-shaped material, only one kind of, or two or more kinds of cells may be used. In one embodiment where there are two or more kinds of cells contained in the bioadhesive sheet-shaped material, the content ratio (purity) of the most abundant cells is 60% or more, preferably 70% or more, and more preferably 75% or more, at the end of the production of the bioadhesive sheet-shaped material.

The cells may be cells derived from heterogeneous cells, or may also be cells derived from homogeneous cells. In this regard, the expression “cells derived from heterogeneous cells” means cells derived from an organism of a species different from that of the recipient to which the bioadhesive sheet-shaped material is attached. For example, in a case where the recipient is a human, cells derived from a monkey or a pig correspond to the cells derived from heterogeneous cells. Further, the expression “cells derived from homogeneous cells” means cells derived from an organism of the same species as that of the recipient. For example, in a case where the recipient is a human, human cells correspond to the cells derived from homogeneous cells. The cells derived from homogeneous cells include self-derived cells (also referred to as “self cells” or “autologous cells”), that is, recipient-derived cells, and cells derived from homogeneous non-self cells (also referred to as “non-autologous cells”). The self-derived cells are advantageous because the self-derived cells do not cause any rejection even when being transplanted. However, cells derived from heterogeneous cells, or cells derived from homogeneous non-self cells can also be used. In a case where such cells derived from heterogeneous cells or derived from homogeneous non-self cells are used, the cells may require immunosuppressive treatment in order to suppress the rejection, in some cases. Cells other than the self-derived cells, that is, cells derived from heterogeneous cells and cells derived from homogeneous non-self cells may also be collectively referred to as “non-self-derived cells”. In one embodiment, the cells are autologous cells or non-autologous cells. In another embodiment, the cells are autologous cells. In other embodiments, the cells are non-autologous cells.

The sheet-shaped cell culture contained in the bioadhesive sheet-shaped material can be produced by any method known to a person skilled in the art (see, for example, JP 6008297 B2, JP 2010-081829 A and JP 2011-110368 A). The method for producing a sheet-shaped cell culture typically includes, but is not limited to, a step of seeding cells on a substrate, a step of forming the seeded cells into a sheet-shaped cell culture, and a step of detaching the formed sheet-shaped cell culture from the substrate. Before the step of seeding cells on a substrate, a step of freezing cells and a step of thawing the cells may be performed. Further, a step of washing the cells may be performed after the step of thawing the cells. Each of these steps can be performed by any known technique suitable for the production of a sheet-shaped cell culture. The step of producing a sheet-shaped cell culture may include 1 or 2 or more of the steps according to the above method for producing a sheet-shaped cell culture as the sub-steps. In one embodiment, a step of proliferating the cells is not included after the step of thawing the cells and before the step of seeding the cells on a substrate.

Examples of the substrate are not particularly limited as long as cells can form a cell culture on the substrate, and include containers made of various materials, and a solid or half-solid surface in a container. The container can have a structure/material that does not allow a liquid such as a liquid culture medium to permeate. Examples of the material include, but are not limited to, polyethylene, polypropylene, Teflon (registered trademark), polyethylene terephthalate, polymethyl methacrylate, nylon-6,6, polyvinyl alcohol, cellulose, silicon, polystyrene, glass, polyacrylamide, polydimethyl acrylamide, and a metal (for example, iron, stainless steel, aluminum, copper, or brass). Further, the container may have at least one flat surface. As such a container, without any limitation, for example, a culture container provided with the bottom made of a substrate capable of forming a cell culture and the liquid-impermeable side can be mentioned. Specific examples of the culture container include, but are not limited to, a cell-culture dish and a cell-culture bottle. The bottom of the container may be transparent or opaque. If the bottom of a container is transparent, cells can be observed and counted from the underside of the container. Further, the container may have a solid or half-solid surface inside thereof. Examples of the solid surface include a plate, and a container, which are made of various materials as described above, and examples of the half-solid surface include a gel and a soft polymer matrix. As the substrate, a substrate prepared by using the above-described materials may be used, or a commercially available material may be used. The substrate, without any limitation, for example, may have an adhesive surface, which is suitable for forming a sheet-shaped cell culture. Specifically, a substrate having a hydrophilic surface, for example, a substrate of which the surface is coated with a hydrophilic compound such as corona discharge-treated polystyrene, a collagen gel, or a hydrophilic polymer, a substrate of which the surface is coated with an extracellular matrix of collagen, fibronectin, laminin, vitronectin, proteoglycan, glycosaminoglycan, or the like, or a cell adhesion factor such as a cadherin family, a selectin family, or an integrin family, or the like can be mentioned. Further, such a substrate is commercially available (for example, Corning (registered trademark) TC-Treated Culture Dish, Corning, or the like). The entirety or part of the substrate may be transparent or opaque.

The surface of the substrate may be coated with a material of which the physical properties change in response to a stimulus, for example, temperature or light. As such a material, without any limitation, a known material, for example, a temperature-responsive material made of a homopolymer or a copolymer of a (meth)acrylamide compound, a N-alkyl-substituted (meth)acrylamide derivative (for example, N-ethyl acrylamide, N-n-propyl acrylamide, N-n-propyl methacrylamide, N-isopropyl acrylamide, N-isopropyl methacrylamide, N-cyclopropyl acrylamide, N-cyclopropyl methacrylamide, N-ethoxyethyl acrylamide, N-ethoxyethyl methacrylamide, N-tetrahydrofurfuryl acrylamide, N-tetrahydrofurfuryl methacrylamide, or the like), a N,N-dialkyl-substituted (meth)acrylamide derivative (for example, N,N-dimethyl (meth)acrylamide, N,N-ethyl methyl acrylamide, N,N-diethyl acrylamide, or the like), a (meth)acrylamide derivative having a cyclic group (for example, 1-(1-oxo-2-propenyl)-pyrrolidine, 1-(1-oxo-2-propenyl)-piperidine, 4-(1-oxo-2-propenyl)-morpholine, 1-(1-oxo-2-methyl-2-propenyl)-pyrrolidine, 1-(1-oxo-2-methyl-2-propenyl)-piperidine, 4-(1-oxo-2-methyl-2-propenyl)-morpholine, or the like), or a vinyl ether derivative (for example, methyl vinyl ether), or a photoresponsive material such as a light-absorbing polymer having an azobenzene group, a copolymer of a vinyl derivative of triphenylmethane leucohydroxide and an acrylamide-based monomer, or N-isopropylacrylamide gel containing spirobenzopyran, may be used (see, for example, JP H02-211865 A, and JP 2003-33177 A). By giving a predetermined stimulus to such a material, the physical properties, for example, the hydrophilicity and the hydrophobicity can be changed, and the detachment of a cell culture attached on the material can be promoted. A culture dish coated with a temperature-responsive material is commercially available (for example, UpCell (registered trademark) of CellSeed Inc., or Cepallet (registered trademark) of DIC Corporation), and such a culture dish can be used in the production methods of the present disclosure.

The substrate may have various shapes, but is preferably flat. Further, the area of the substrate is not particularly limited, and may be, for example, around 1 cm² to around 200 cm², around 2 cm² to around 100 cm², or around 3 cm² to around 50 cm². For example, as the substrate, a circular culture dish having a diameter of 10 cm can be mentioned. In this case, the area is 56.7 cm².

The substrate may be coated with serum. By using a substrate coated with serum, a sheet-shaped cell culture with a higher density can be formed. The expression “coated with serum” means a state that a serum component adheres onto a surface of the substrate. Such a state can be obtained, for example, by processing a substrate with serum, without any limitation. The processing with serum includes bringing serum into contact with a substrate, and performing the incubation for a predetermined period of time as needed.

As the serum, heterologous serum and/or homologous serum can be used. In a case where a bioadhesive sheet-shaped material is used by the attachment, the heterologous serum means serum derived from an organism of a species different from that of the recipient. For example, in a case where the recipient is a human, serum derived from a bovine or a horse, for example, fetal bovine serum/fetal calf serum (FBS/FCS), calf serum (CS), horse serum (HS), or the like corresponds to the heterologous serum. Further, the expression “homologous serum” means serum derived from an organism of the same species as that of the recipient. For example, in a case where the recipient is a human, human serum corresponds to the homologous serum. The homologous serum includes self serum (also referred to as “autologous serum”), that is, serum derived from the recipient, and homologous non-autologous serum derived from an individual of the same species other than the recipient. Serum other than self serum, that is, heterologous serum and homologous non-autologous serum may be collectively referred to as “non-self serum”.

The serum for coating on a substrate is commercially available, or can be prepared from the blood collected from a desired organism by a conventional method. Specifically, for example, a method in which the collected blood is left to stand at room temperature for around 20 minutes to around 60 minutes so as to be coagulated, the coagulated blood is centrifuged at around 1000×g to around 1200×g, and a supernatant is collected, or the like can be mentioned.

In a case where the incubation is performed on a substrate, serum may be used in undiluted form, or may be diluted for use. The serum can be diluted, without any limitation, in any medium, for example, water, a saline solution, various buffer solutions (for example, PBS, HBSS and the like), various liquid media (for example, DMEM, MEM, F12, DMEM/F12, DME, RPM11640, MCDB (such as MCDB102, 104, 107, 120, 131, 153, or 199), L15, SkBM, or RITC80-7) or the like. The dilution concentration is not particularly limited as long as the serum component can adhere onto a substrate, and is, for example, around 0.5% to around 100% (v/v), preferably around 1% to around 60% (v/v), and more preferably around 5% to around 40% (v/v).

The incubation time is also not particularly limited as long as the serum component can adhere onto a substrate, and is, for example, around 1 hour to around 72 hours, preferably around 2 hours to around 48 hours, more preferably around 2 hours to around for 24 hours, and furthermore preferably around 2 hours to around 12 hours. The incubation temperature is also not particularly limited as long as the serum component can adhere onto a substrate, and is, for example, around 0° C. to around 60° C., preferably around 4° C. to around 45° C., and more preferably room temperature to around 40° C.

The serum may be discarded after incubation. As the technique for discarding the serum, suction with a pipette or the like, or a conventionally-used technique for discarding liquid such as decantation can be used. In a preferred embodiment, after the serum is discarded, the substrate may be washed with a serum-free washing solution. The serum-free washing solution is not particularly limited as long as it is a liquid medium that does not contain serum and does not adversely affect the serum component adhered onto a substrate, and the washing can be performed, without any limitation, for example, by water, a saline solution, various buffer solutions (for example, PBS, HBSS and the like), various liquid media (for example, DMEM, MEM, F12, DMEM/F12, DME, RPM11640, MCDB (such as MCDB102, 104, 107, 120, 131, 153, or 199), L15, SkBM, or RITC80-7), or the like. As the washing technique, without any limitation, a conventionally-used technique for washing a substrate, for example, a technique in which a serum-free washing solution is added onto a substrate, stirred for a predetermined time (for example, around 5 seconds to around 60 seconds), and then discarded, or the like can be used.

The substrate may be coated with a growth factor. In this regard, the expression “growth factor” means any substance that promotes proliferation of cells as compared with a substrate that is not coated with the growth factor, and examples of the growth factor include epidermal growth factor (EGF), vascular endothelial growth factor (VEGF), and fibroblast growth factor (FGF). The technique for coating on a substrate with a growth factor, the discarding technique, and the washing technique are basically the same as those of serum except that the dilution concentration at the time of incubation is, for example, around 0.0001 μg/mL to around 1 μg/mL, preferably around 0.0005 μg/mL to around 0.05 μg/mL, and more preferably around 0.001 μg/mL to around 0.01 μg/mL.

The substrate may be coated with a steroid drug. In this regard, the expression “steroid drug” refers to a compound that could exert an adverse effect on the living body, such as adrenocortical insufficiency, or Cushing syndrome, among the compounds having a steroid nucleus. Examples of the compound include, but are not limited to, cortisol, prednisolone, triamcinolone, dexamethasone, and betamethasone. The technique for coating on a substrate with a steroid drug, the discarding technique, and the washing technique are basically the same as those of serum except that the dilution concentration at the time of incubation is, for example, 0.1 μg/mL to around 100 μg/mL, preferably around 0.4 μg/mL to around 40 μg/mL, and more preferably around 1 μg/mL to around 10 μg/mL, as dexamethasone.

The substrate may be coated with any one of the serum, the growth factor, and the steroid drug, or may be coated with any combination of the serum, the growth factor, and the steroid drug, that is, a combination of the serum and the growth factor, a combination of the serum and the steroid drug, a combination of the serum, the growth factor, and the steroid drug, or a combination of the growth factor and the steroid drug. In a case of coating with multiple components, the coating with these components may be performed at the same time by mixing the components with each other, or may be performed in separate steps.

The substrate may be seeded with cells immediately after being coated with serum and the like, or may also be stored after being coated and then seeded with cells. The coated substrate can be stored for a long period of time, for example, by keeping the substrate at around 4° C. or less, preferably around −20° C. or less, and more preferably around −80° C. or less.

Seeding of cells on a substrate can be performed by any known technique under any known conditions. The seeding of cells on a substrate may be performed, for example, by injecting a cell suspension in which cells are suspended in a liquid culture medium into a substrate (culture container). For the injection of the cell suspension, an instrument suitable for the injection operation of the cell suspension, such as a dropper or a pipette, can be used.

In one embodiment, the seeding can be performed at a density of around 7.1×10⁵ cells/cm² to around 3.0×10⁶ cells/cm², around 7.3×10⁵ cells/cm² to around 2.8×10⁶ cells/cm², around 7.5×10⁵ cells/cm² to around 2.5×10⁶ cells/cm², around 7.8×10⁵ cells/cm² to around 2.3×10⁶ cells/cm², around 8.0×10⁵ cells/cm² to around 2.0×10⁶ cells/cm², around 8.5×10⁵ cells/cm² to around 1.8×10⁶ cells/cm², around 9.0×10⁵ cells/cm² to around 1.6×10⁶ cells/cm², around 1.0×10⁶ cells/cm² to around 1.6×10⁶ cells/cm², or the like.

The expression “biodegradable gel layer” refers to a layer formed of biodegradable gel that is a gel being degraded in the body, absorbed into the body, metabolized, and excreted. Examples of the biodegradable gel include, but are not limited to, a fibrin gel, and a gel formed by Adspray (registered trademark) (manufactured by TERUMO CORPORATION). The biodegradable gel refers to a gel that generates viscosity by mixing preferably two kinds of liquids. For example, the fibrin gel is a gel having high strength formed by the action of thrombin on fibrinogen by mixing a liquid containing the fibrinogen (hereinafter, referred to as “fibrinogen solution”) with a liquid containing the thrombin (hereinafter, referred to as “thrombin solution”).

Further, since the biodegradable gel can be degraded in the body, the bioadhesive sheet-shaped material can be used for the attachment to an organ of a subject together with the contained biodegradable gel layer. In the present disclosure, the thickness of the biodegradable gel layer is 5 μm to 1300 μm, preferably 50 μm to 300 μm.

In one embodiment, the thickness of the biodegradable gel layer is uniform. In this regard, the term “uniform” means that the difference between the thickest part and the thinnest part is 20% or less, preferably 10% or less, and more preferably 5% or less.

The bioadhesive sheet-shaped material of the present disclosure is used by attaching an extracellular matrix layer contained in the bioadhesive sheet-shaped material onto a surface of an organ. The organ to which the bioadhesive sheet-shaped material is attached may be an organ of any individual organism, but is preferably an animal organ, more preferably a mammalian organ, and furthermore preferably a human organ. The term “organ” refers to an organ having specific morphology and function, which is within the body cavity, and examples of the organ include a digestive organ such as liver, gallbladder, large intestine, stomach, or duodenum; a circulatory organ such as heart, or spleen; a respiratory organ such as lung; a urinary organ such as kidney; a genital organ such as prostate, uterus, or ovary; and an endocrine organ such as adrenal gland. It is considered that oxygen, nutrients, and the like need to be supplied to a sheet-shaped cell culture in order to engraft an organ with the sheet-shaped cell culture and to retain the organ in a favorable condition, and since the bioadhesive sheet-shaped material contains an extracellular matrix layer, it is considered that the bioadhesive sheet-shaped material releases more cytokines such as VEGF and HGF, which have angiogenic activity.

In one embodiment, the attachment of the bioadhesive sheet-shaped material onto a surface of an organ is performed under a laparoscope or a thoracoscope. In this regard, the expression “attachment under a laparoscope or a thoracoscope” means that the bioadhesive sheet-shaped material is delivered to the vicinity of an organ of a subject through a path of an instrument (for example, a port for a laparoscope, a port for a thoracoscope, or the like) prepared for laparoscopic or thoracoscopic surgery in a subject, and attached onto a surface of the organ. When the attachment is performed under a laparoscope or a thoracoscope, the bioadhesive sheet-shaped material may be curved with forceps or the like, and the curved bioadhesive sheet-shaped material may be passed through a path of an instrument, and spread in the vicinity of an organ so as to be attached to the organ. Further, when the bioadhesive sheet-shaped material is passed through a path of an instrument in this way, a device for delivery may be used. As the device for delivery, for example, a therapeutic-substance carrying/administering appliance (see, JP 2009-000511 A), or the like can be used, although the device is not limited to such an appliance.

Accordingly, in one embodiment, the organ to which the bioadhesive sheet-shaped material is attached is an organ that can be subjected to laparoscopic surgery or thoracoscopic surgery, that is, an organ in the body, capable of being approached from outside the body by a laparoscope or a thoracoscope, and examples of the organ include, but are not limited to, liver, gallbladder, spleen, large intestine, stomach, duodenum, prostate, kidney, adrenal gland, uterus, ovary, and heart. Since there may be a case where the bioadhesive sheet-shaped material adheres to the abdominal wall in small intestine, the organ to which the bioadhesive sheet-shaped material is attached is preferably an organ selected from the group consisting of stomach, large intestine, duodenum, and liver.

The cells contained in the bioadhesive sheet-shaped material forming the sheet-shaped cell culture are not particularly limited, as long as they can produce an extracellular matrix and include, for example, adherent cells (adhesion cells). Examples of the adherent cells include adhesive somatic cells (for example, cardiomyocytes, fibroblast cells, epithelial cells, endothelial cells, liver cells, pancreatic cells, kidney cells, adrenal cells, periodontal ligament cells, gingival cells, periosteal cells, skin cells, synovial cells, and chondrocytes), and stem cells (for example, tissue stem cells such as myoblasts, and heart stem cells, pluripotent stem cells such as embryonic stem cells, and induced pluripotent stem (iPS) cells, and mesenchymal stem cells). The somatic cells may be stem cells, particularly cells differentiated from iPS cells (iPS cell-derived adherent cells). Un-limited examples of the cells contained in the cell culture include myoblast cells (for example, skeletal myoblast cells), mesenchymal stem cells (for example, bone marrow, adipose tissue, peripheral blood, skin, hair root, muscle tissue, endometrium, placenta, and umbilical cord blood-derived cells), cardiomyocytes, mesothelial cells, stromal cells (for example, fibroblast cells, and adipose-derived cells), alveolar tissue cells, cardiac stem cells, embryonic stem cells, iPS cells, synovial cells, chondrocytes, epithelial cells (for example, small intestinal epithelial cells, oral mucosal epithelial cells, retinal pigment epithelial cells, and nasal epithelial cells), endothelial cells (for example, vascular endothelial cells), liver cells (for example, liver parenchymal cells, and liver progenitor cells), pancreatic cells (for example, pancreatic islet cells, and pancreatic progenitor cells), kidney cells, adrenal cells, periodontal ligament cells, gingival cells, periosteal cells, immune cells (T cells, NK cells), and skin cells (for example, epidermal keratinocytes). In this regard, the iPS cells are cells having pluripotency and replication competence, which are induced by introducing a gene. Un-limited examples of the iPS cell-derived adherent cells include iPS cell-derived cardiomyocytes, fibroblast cells, epithelial cells, endothelial cells, liver cells, pancreatic cells, kidney cells, adrenal cells, periodontal ligament cells, gingival cells, periosteal cells, skin cells, synovial cells, and chondrocytes. In some embodiments, the cells contained in the bioadhesive sheet-shaped material are preferably mesenchymal stem cells or myoblast cells, more preferably myoblast cells.

Myoblast cells are well known in the technical field to which the present disclosure belongs, and can be prepared from skeletal muscle by any known method (for example, methods disclosed in JP 2007-89442 A), and as the myoblast cells, for example, catalog number: CC-2580 of Lonza Japan, product code 3520 of Cosmo Bio Co., Ltd., or the like can be obtained commercially. The myoblast cells are not limited to such cells, and can be identified by a marker such as CD56, α7 integrin, myosin heavy chain IIa, myosin heavy chain IIb, myosin heavy chain IId (IIx), MyoD, Myf5, Myf6, myogenin, desmin, or PAX3. In one embodiment, the myoblast cells are CD56 positive. In one embodiment, the myoblast cells are CD56 positive and desmin positive.

In a case where the myoblast cells are prepared from striated muscle tissue, the prepared cell population contains fibroblast cells. When the cell culture according to the present disclosure is produced, in a case where a cell population containing the myoblast cells prepared from striated muscle tissue is used, a certain amount of fibroblast cells is contained in the cell population. The fibroblast cells are well known in the technical field to which the present disclosure belongs, and can be identified by a marker such as TE-7 (see, for example, Rosendaal et al., J Cell Sci. 1994, 107(Pt1): 29-37, Goodpaster et al. J Histochem Cytochem. 2008, 56(4): 347-358).

In one embodiment, the cells forming the cell culture include myoblast cells prepared from striated muscle tissue. Accordingly, the cell population used in production of the cell culture can contain myoblast cells and fibroblast cells. In one embodiment, the cell population used in production of the cell culture can have a CD56-positive rate of 50% or more, 60% or more, 65% or more, 70% or more, 75% or more, 80% or more, 85% or more, or 90% or more, preferably 60% or more.

In a case where the bioadhesive sheet-shaped material contains myoblast cells, the cell population used in production of the bioadhesive sheet-shaped material can include fibroblast cells, but in a case where the content of the fibroblast cells is extremely high, the content of myoblast cells is lowered, and thus, this is not preferable. Accordingly, in one embodiment, the cell population used in production of the cell culture can have a TE-7 positive rate of 50% or less, 40% or less, 35% or less, 30% or less, 25% or less, 20% or less, 15% or less, or 10% or less, preferably 40% or less.

The cell population used in production of the bioadhesive sheet-shaped material can contain cells other than the myoblast cells and the fibroblast cells, and the smaller the number such cells are, the more preferable the cell population is. Accordingly, the higher the total value of the CD56-positive rate and the TE-7 positive rate is, the more preferable the cell population is, and the total value can be, for example, 80% or more, 85% or more, 90% or more, 95% or more, 96% or more, 97% or more, 98% or more, or 99% or more, preferably 90% or more.

In one embodiment, the bioadhesive sheet-shaped material further contains a medical component. In this regard, the expression “medical component” means any component that treats abnormalities of an organ engrafted with the bioadhesive sheet-shaped material. Examples of the abnormalities of an organ include, but are not limited to, inflammation generated in an organ, tumor formed in an organ, and ulcer formed in an organ, and examples of the medical component to treat the abnormalities of such an organ include, but are not limited to, an anti-inflammatory agent, an antimicrobe agent, an antifungal agent, an antihistamine, an adrenal cortical hormone, an anticancer agent, and any combination thereof.

In addition to an extracellular matrix layer, a sheet-shaped cell culture, a biodegradable gel layer, and/or a medical component, the bioadhesive sheet-shaped material may contain various additional components, for example, a pharmaceutically acceptable carrier, a component that enhances the survivability, bioadhesiveness and function of the bioadhesive sheet-shaped material, and a component that assists the medical component useful for treatment of abnormalities of an organ of a subject. As such additional components, any known additional components can be used, and a person skilled in the art is well-versed in the additional components. Further, the bioadhesive sheet-shaped material can be used in combination with the component that enhances the survivability, bioadhesiveness and function, and/or the like of the bioadhesive sheet-shaped material, and/or the component that assists the medical component useful for treatment of abnormalities of an organ of a subject, and the like.

The present disclosure also provides a method for producing a bioadhesive sheet-shaped material, the method comprising: a step of seeding cells on a substrate; a step of forming the seeded cells into a sheet shape; and a step of detaching the formed sheet-shaped cell culture from the substrate.

The sheet-shaped cell culture contained in the bioadhesive sheet-shaped material can be produced by any method known to a person skilled in the art (see, for example, JP 6008297 B2, JP 2010-081829 A, or JP 2011-110368 A) as described above. The method for producing a sheet-shaped cell culture typically includes, but is not limited to, a step of seeding cells on a substrate, a step of forming the seeded cells into a sheet-shaped cell culture, and a step of detaching the formed sheet-shaped cell culture from the substrate.

The method for producing a bioadhesive sheet-shaped material may further include a step of forming an extracellular matrix layer. Although depending on various conditions, an extracellular matrix forming an extracellular matrix layer can be produced, for example, by seeding cells, and then culturing the seeded cells contained in a sheet-shaped cell culture for 24 hours or more, for example, for 24 hours, 30 hours, 36 hours, 42 hours, 48 hours, 54 hours, 60 hours, 66 hours, or 72 hours. The produced extracellular matrix is formed in a layer form, for example, between the sheet-shaped cell culture and a substrate. In this case, in the subsequent step of detaching the formed sheet-shaped cell culture from the substrate, the sheet-shaped cell culture is preferably detached from the substrate without damaging the formed extracellular matrix layer. The method for detaching the sheet-shaped cell culture from the substrate so as not to damage the formed extracellular matrix layer includes, but is not limited to, using a culture dish coated with a temperature-responsive material for the culture.

The method for producing a bioadhesive sheet-shaped material disclosed here may further includes a step of forming a biodegradable gel layer. The method for forming a biodegradable gel layer can include, for example, but is not limited to, a step of forming a fibrin gel layer by adding a fibrinogen solution dropwise onto a sheet-shaped cell culture, and then spraying a thrombin solution (see, JP 6495603 B2), or the like can be mentioned. The method for producing a bioadhesive sheet-shaped material may further include a step of adding a medical component. The step of adding a medical component may be performed before or after the step of forming the seeded cells into a sheet shape, or may be performed after the step of forming the biodegradable gel layer.

Further, another aspect of the present disclosure provides a method for treating a disease that is ameliorated by application of the bioadhesive sheet-shaped material, the method including attaching a surface having an extracellular matrix layer of the bioadhesive sheet-shaped material in an effective amount (effective amount of the bioadhesive sheet-shaped material) to an organ of a subject in need thereof.

The term “subject” means any individual organism, preferably an animal, more preferably a mammal, and furthermore preferably an individual human.

Further, the term “treatment” includes cure of disease, and all types of medically acceptable prophylactic and/or therapeutic interventions for the purpose of temporary remission, prophylaxis or the like. For example, the term “treatment” includes medically acceptable intervention for the various purposes, including retardation or suspension of the progression of disease, regression or disappearance of lesion, prevention of the onset or recurrence of the disease, and the like.

The term “effective amount” means, for example, an amount (for example, the number of the cells contained in a bioadhesive sheet-shaped material, the size, weight, or the like of the sheet-shaped cell culture contained in a bioadhesive sheet-shaped material) with which the onset or recurrence of disease can be suppressed, the symptoms can be alleviated, or the progression can be retarded or suspended, and preferably an amount with which the onset or recurrence of disease is prevented, or the disease is cured. Further, an effective amount can include an amount with which any adverse effect exceeding the benefit of administration is not exerted. Such an amount can be appropriately determined by, for example, a test or the like in an experimental animal or a disease model animal such as a mouse, a rat, a dog, or a pig, and such a test method is well known to a person skilled in the art. In addition, the size of the histological lesion to be treated can be an important indicator for determining the effective amount.

In accordance with the production method of the present disclosure, the treatment method may further include a step of producing a bioadhesive sheet-shaped material. The treatment method of the present disclosure may further include a step of collecting cells (for example, skin cells, blood cells, or the like in a case of using iPS cells) for producing a sheet-shaped cell culture contained in a bioadhesive sheet-shaped material, or tissue (for example, skin tissue, blood, or the like in a case of using iPS cells) that is a supply source of cells, from a subject before the step of producing the bioadhesive sheet-shaped material. In one embodiment, a subject from which the cells or the tissue to be a supply source of cells is collected is the same individual as a subject to which a bioadhesive sheet-shaped material is attached. In another embodiment, a subject from which the cells or the tissue to be a supply source of cells is collected is another individual of the same species as that of a subject to which a bioadhesive sheet-shaped material is attached. Further, in another embodiment, a subject from which the cells or the tissue to be a supply source of cells is collected is an individual of a species different from that of a subject to which a bioadhesive sheet-shaped material is attached.

As the attachment method, typically, direct attachment to tissue can be mentioned, and for example, direct attachment to an organ of a subject to be attached. The method for attaching the bioadhesive sheet-shaped material may be, for example, attachment under a laparoscope or a thoracoscope, in addition to the above attachments. In this regard, the expression “attachment under a laparoscope or a thoracoscope” means that the bioadhesive sheet-shaped material is delivered to the vicinity of an organ of a subject through a path of an instrument (for example, a port for a laparoscope, a port for a thoracoscope, or the like) prepared for laparoscopic or thoracoscopic surgery, and attached onto a surface of an organ of a subject, and such delivery to the vicinity of an organ of a subject may be performed by using a device for delivery. The device for delivery may be, for example, a device including a support member for supporting a bioadhesive sheet-shaped material, and a protective member with a low coefficient of friction for protecting one surface of the support, in which the support supporting the bioadhesive sheet-shaped material is protected with the protective member, and the protected support is rolled up so as to be inserted into a cylindrical body.

### EXAMPLES

The present disclosure will be described in more detail with reference to the following Examples, which show specific examples within the present disclosure. The present disclosure is not limited to these specific examples.

### Example 1: Preparation of Sheet-Shaped Cell Culture

The striated muscle in the lower limb of a pig is collected under general anesthesia, and the collected tissue is processed with an enzymatic digestive juice containing collagenase and trypsin, and dispersed in single cells. Such cells were cultured in a 20% FBS-containing MCDB131 medium under the conditions of 37° C. and 5% CO₂ until the confluency was obtained, and the cells were collected. 2.2×10⁷ collected cells were seeded in a 60-mm temperature-responsive culture dish (UpCell (registered trademark), 6-cm dish, CS3006, CellSeed Inc.), cultured in a 20% FBS-containing DMEM/F12 medium for 12 hours, and the sheet-forming and the forming of an extracellular matrix layer were performed. After that, by lowering the temperature to 20° C., a sheet-shaped cell culture was detached and collected from the culture dish while having the extracellular matrix layer. Onto a surface on the side opposite to the surface having the extracellular matrix layer of the sheet-shaped cell culture, 500 μL of fibrinogen solution (in which a contained material (lyophilized fibrinogen powder) of Vial 1 of Bolheal (registered trademark) tissue adhesion bond (Teijin Pharma Limited.) was dissolved with a contained solution (fibrinogen dissolving solution) of Vial 2 at a fibrinogen concentration of 80 mg/mL) was added dropwise, and then 800 μL of thrombin solution (in which a contained material (lyophilized thrombin powder) of Vial 3 of Bolheal (registered trademark) tissue adhesion bond (Teijin Pharma Limited.) was dissolved with a contained solution (thrombin dissolving solution) of Vial 4 at a thrombin concentration of 250 unit/mL) was sprayed. By the reaction in the dropwise addition of the fibrinogen solution and the subsequent spraying of the thrombin solution, a biodegradable gel layer made of fibrin gel was formed on a surface on the side opposite to the surface having an extracellular matrix layer of the sheet-shaped cell culture, and a bioadhesive sheet-shaped material was obtained.

### Example 2: Attachment of Bioadhesive Sheet-Shaped Material to Organ and Collection of Tissue

A bioadhesive sheet-shaped material prepared in Example 1 was attached under general anesthesia to a pig from which the striated muscle had been collected in Example 1. The attachment was performed so as to attach a surface of an extracellular matrix layer of the bioadhesive sheet-shaped material onto a surface of the stomach, the large intestine, or the liver. The pig to which the bioadhesive sheet-shaped material had been attached was subjected to laparotomy again under general anesthesia on day 3 after the attachment, the attachment site was observed, and then the tissue was collected.

### Example 3: Observation of Stained Tissue Image of Collected Tissue

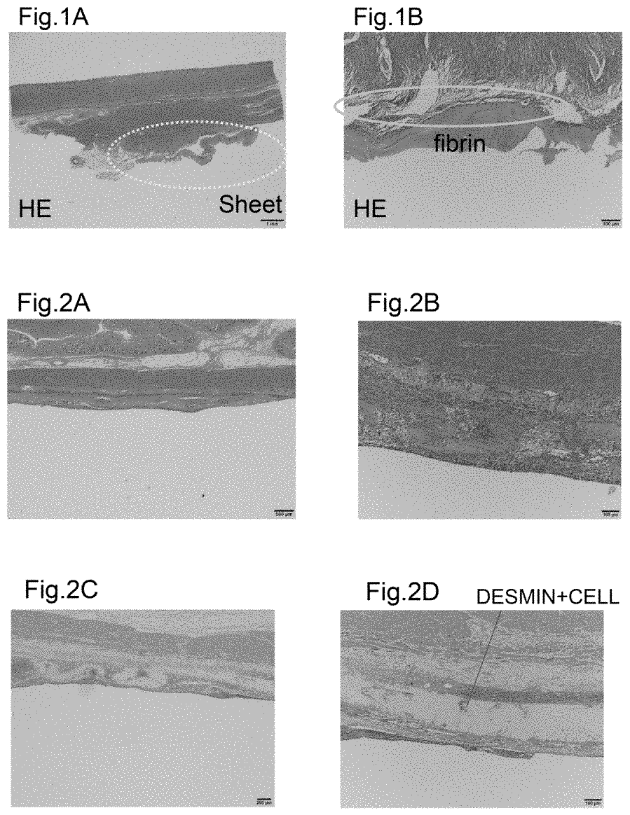

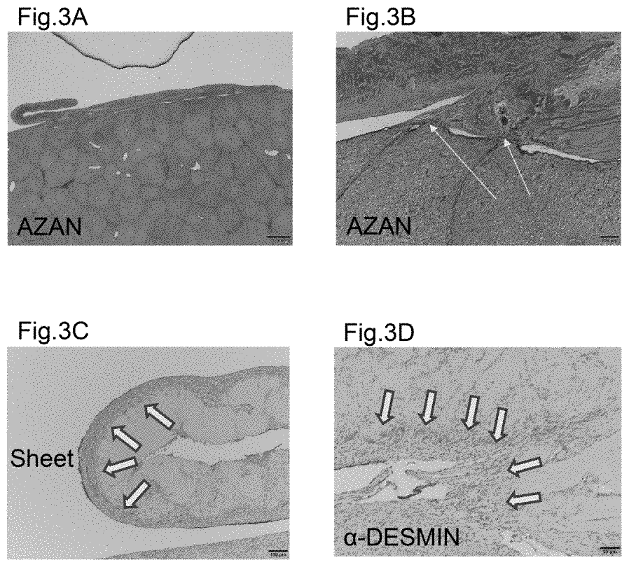

Each stained tissue image was obtained from the collected tissue. Stained tissue images that were obtained by staining the tissue from the stomach with hematoxylin-eosin staining are shown in FIGS. 1A and 1B. Stained tissue images that were obtained by staining the tissue from the large intestine with hematoxylin-eosin staining are shown in FIGS. 2A and 2B, and stained tissue images that were obtained by immunostaining the tissue from the large intestine with the use of an anti-desmin antibody are shown in FIGS. 2C and 2D. Stained tissue images that were obtained by staining the tissue from the liver with Azan are shown in FIGS. 3A and 3B, and stained tissue images that were obtained by immunostaining the tissue from the liver with the use of an anti-desmin antibody are shown in FIGS. 3C and 3D.

It has been confirmed from the stained tissue images of FIGS. 1A to 3D that the bioadhesive sheet-shaped material is engrafted into an organ of a subject to be attached, and the organ and the bioadhesive sheet-shaped material are histologically continuous with each other, in the stomach, the large intestine, and the liver. In particular, in the part surrounded by a circle with a straight line in FIG. 1B, and the parts indicated by arrows in FIGS. 3B, 3C and 3D, it has been remarkably confirmed that the organ and the bioadhesive sheet-shaped material are histologically continuous with each other.

The detailed description above describes embodiments of a bioadhesive sheet-shaped material, a method for producing the bioadhesive sheet-shaped material, and a method for treating a disease by using the bioadhesive sheet-shaped material. These disclosed embodiments represent examples of the bioadhesive sheet-shaped material, production method and treatment method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A bioadhesive sheet-shaped material that is configured to be attached onto a surface of an organ, the bioadhesive sheet-shaped material comprising:
an extracellular matrix layer;
a sheet-shaped cell culture;
a biodegradable gel layer,
the extracellular matrix layer being one surface of the bioadhesive sheet-shaped material, and the biodegradable gel layer being an opposite surface of the bioadhesive sheet-shaped material, with the sheet-shaped cell culture being interposed between the extracellular matrix layer and the biodegradable gel layer; and
the extracellular matrix layer being the surface of the bioadhesive sheet-shaped material that is to be attached onto the surface of the organ.

2. The bioadhesive sheet-shaped material according to claim 1, wherein the extracellular matrix having a thickness from 1 nm to 100 nm.

3. The bioadhesive sheet-shaped material according to claim 1, wherein the biodegradable gel layer has a thickness of 5 μm to 1300 μm.

4. The bioadhesive sheet-shaped material according to claim 1, wherein the sheet-shaped cell culture contains somatic cells.

5. The bioadhesive sheet-shaped material according to claim 4, wherein the somatic cells are myoblast cells or mesenchymal stem cells.

6. The bioadhesive sheet-shaped material according to claim 1, wherein a difference between a thickest part and a thinnest part of the biodegradable gel layer is 20% or less.

7. The bioadhesive sheet-shaped material according to claim 1, wherein the bioadhesive sheet-shaped material further comprises a medical component.

8. The bioadhesive sheet-shaped material according to claim 7, wherein the medical component is an anti-inflammatory agent, an antimicrobe agent, an antifungal agent, an antihistamine, an adrenal cortical hormone, an anticancer agent, and any combination thereof.

9. The bioadhesive sheet-shaped material according to claim 7, wherein the bioadhesive sheet-shaped material further comprises a pharmaceutically acceptable carrier, a component that enhances the survivability, bioadhesiveness and function of the bioadhesive sheet-shaped material, a component that assists the medical component in treatment of abnormalities of an organ of a subject, or any combination thereof.

10. A method for producing a bioadhesive sheet-shaped material that is configured to be attached onto a surface of an organ, the method comprising:
seeding cells on a substrate;
forming the seeded cells into a sheet shape so that a formed sheet-shaped cell culture exists on the substrate;
detaching the formed sheet-shaped cell culture from the substrate; and
positioning the formed sheet-shaped cell culture detached from the substrate between an extracellular matrix layer and a biodegradable gel layer so that the extracellular matrix layer is on surface of the bioadhesive sheet-shaped material and the biodegradable gel layer is an opposite surface of the bioadhesive sheet-shaped material, with the extracellular matrix layer being the surface of the bioadhesive sheet-shaped material that is to be attached onto the surface of the organ.

11. The method according to claim 10, wherein the substrate in which the cells are seeded is coated with a serum, a growth factor, a steroid drug, or any combination of the serum, the growth factor, and the steroid drug.

12. The method according to claim 10, further comprising adding a medical component to the bioadhesive sheet-shaped material.

13. The method according to claim 12, wherein the medical component is an anti-inflammatory agent, an antimicrobe agent, an antifungal agent, an antihistamine, an adrenal cortical hormone, an anticancer agent, and any combination thereof.

14. The method according to claim 10, further comprising adding to the bioadhesive sheet-shaped material: i) a pharmaceutically acceptable carrier; ii) a component that enhances the survivability, bioadhesiveness and function of the bioadhesive sheet-shaped material; iii) a component that assists the medical component in treatment of abnormalities of an organ of a subject; or iv) any combination thereof.

15. A method for treating a disease of a subject that is ameliorated by application of a bioadhesive sheet-shaped material, the method comprising:
attaching the bioadhesive sheet-shaped material to an organ of the subject, the bioadhesive sheet-shaped material comprising an extracellular matrix layer, a sheet-shaped cell culture and a biodegradable gel layer, the extracellular matrix layer being one surface of the bioadhesive sheet-shaped material and the biodegradable gel layer being an opposite surface of the bioadhesive sheet-shaped material, with the sheet-shaped cell culture being interposed between the extracellular matrix layer and the biodegradable gel layer; and
the attaching of the bioadhesive sheet-shaped material to the organ of the subject including attaching the extracellular matrix layer of the bioadhesive sheet-shaped material in an effective amount to the organ of the subject.

16. The method according to claim 15, wherein the attaching of the bioadhesive sheet-shaped material to the organ of the subject is performed under a laparoscope or under a thoracoscope.

17. The method according to claim 15, wherein
the attaching of the bioadhesive sheet-shaped material to the organ of the subject is performed by using a device that includes:
a support member on which is supported the bioadhesive sheet-shaped material; and
a protective member with a low coefficient of friction for protecting one surface of the support, wherein
the support supporting the bioadhesive sheet-shaped material is protected with the protective member, the protected support is rolled up, and the rolled-up support is inserted into a cylindrical body.

18. The method according to claim 17, wherein the subject is a human body and the organ is an organ in the human body, the organ in the human body being a digestive organ, a circulatory organ, a respiratory organ, a urinary organ, a genital organ or an endocrine organ.

19. The method according to claim 17, wherein the subject is a human body and the organ is an organ in the human body, the organ in the human body being liver, gallbladder, spleen, large intestine, stomach, duodenum, prostate, kidney, adrenal gland, uterus, ovary, or heart.

20. The method according to claim 17, wherein the bioadhesive sheet-shaped material also includes: i) a pharmaceutically acceptable carrier; ii) a component that enhances the survivability, bioadhesiveness and function of the bioadhesive sheet-shaped material; iii) a component that assists the medical component in treatment of abnormalities of an organ of a subject; or iv) any combination thereof.
